(19) United States
(12) Patent Application Publication
(10) Pub. No.: US 2019/0343756 A1
(43) Pub. Date: Nov. 14, 2019

(54) SORGHUM DISTILLERY RESIDUE EXTRACT FOR APPLICATION IN COSMETIC PRODUCTS

(71) Applicant: HAIR O'RIGHT INTERNATIONAL CORPORATION, TAOYUAN (TW)

(72) Inventors: WANG-PING KO, TAOYUAN (TW); YUN-TING WU, KAOHSIUNG (TW)

(21) Appl. No.: 15/976,564

(22) Filed: May 10, 2018

(51) Int. Cl. A61K 8/9794; A61Q 19/02; B01D 11/02

(52) U.S. Cl. CPC A61K 8/9794; B01D 11/0288; B01D 11/0203; A61Q 19/02

(57) ABSTRACT

The invention discloses a sorghum distillery residue extract, using Kinmen sorghum distillery residue as raw material and obtained by extracting from the sorghum distillery residue with pure water and a supercritical fluid. The sorghum distillery residue extract has antioxidative activity, ability to inhibit tyrosinase, and ability to reduce the content of melanin produced, and can be used as an additive for preparation of cosmetic or cosmeceutical compositions. The sorghum distillery residue extract has better cytotoxicity, antioxidative capacity and whitening ability when being in an amount of 0.001 weight percent to 0.003 weight percent, based on a total weight of total polyphenols, from the distillery residue, of the compositions.

### FIELD OF THE INVENTION

The present invention relates to a sorghum distillery residue extract, and more particularly to a sorghum distillery residue extract that can be used as an additive for preparation of cosmetic or cosmeceutical compositions.

### BACKGROUND OF THE INVENTION

Sorghum distillery residue (SDR) is a byproduct of sorghum liquor brewing. Approximately 8.2 kg of sorghum distillery residue can be produced after liquor is brewed with 25 kg of sorghum. Daily amounts of sorghum distillery residue produced at Kinmen Kaoliang Liquor Inc. (Taiwan) reach up to 250 metric tons. Currently, the SDR is mainly provided as livestock feeds in Kinmen (Taiwan), or as organic fertilizers for general family farms or small farms. However, the amount of above-mentioned usage is still too low. The amount of the SDR that can be consumed is quite limited. Thus most of the SDR is not used and is treated as industrial waste. Also, a production process of general sorghum liquor is with three-time distillation and three-time brewing, wherein a biomass utilization ratio is about 90%. However, the production process of the sorghum liquor of Kinmen Kaoliang Liquor Inc. (Taiwan) is with two-time distillation and two-time brewing, wherein the biomass utilization ratio is less than 70%. Therefore, it is expected that functional ingredients of this substance can be extracted by a green and nontoxic process, and can be applied to compositions of beauty care products.

According to research at Kinmen Kaoliang Liquor Inc. (2001), in distillery residue byproducts after sorghum fermentation, the amounts of starch can still reach more than 25% (dry weight). Some literatures indicate that sorghum has polyphenol compounds, with rich biological activity and pharmacological activity, such as tannins, flavonoids, phenolic acid, and other functional ingredients (e.g. policosanols and phytochemicals). Studies show that the functional ingredients have functions such as free radical scavenging in vivo, anti-lipid oxidation, cardiovascular diseases prevention, human serum cholesterol decreases, anticancer, and antibacterial. There are also studies showing that mullets with intake of feeds added with sorghum distillery residue can improve anti-oxidation in blood and can decrease blood coagulation to have characteristics of cold resistance for improving growth rate in winter.

The present invention further provides a fully green and nontoxic extraction process without harmful chemical solvents. Also, the present invention discloses an optimal ratio of an additive best suitable for application in the preparation of cosmetic or cosmeceutical compositions in the present process.

### SUMMARY OF THE INVENTION

In addition to a solvent extraction method (using solvents and pure water at different temperatures) for extraction from sorghum distillery residue, the present invention also uses supercritical carbon dioxide fluid extraction equipment for extraction. The method of processes of the present invention is applied to the extraction of functional ingredients of skin care products. Therefore, considering the requirements of compositions that can be applied to skin care products, when using the solvent extraction method, it is necessary to avoid the use of organic solvents, which may cause harm to the environment and human body, such as methanol and hexane. Pure water is chosen. The extraction is conducted by regulating ratios and temperatures thereof.

In addition to the use of the solvent extraction method which is a common method, the supercritical carbon dioxide fluid extraction equipment is also used. Using carbon dioxide as a fluid of the supercritical extraction meets the standard of the green industrial process, and also fulfills CSR (Corporate Social Responsibility). Also, it meets each nation's health regulation about cosmetic and skincare products for being without residual substances harmful to the human body. Bioactive ingredients and the like in extracts can be better reserved due to low extraction temperature. Furthermore, due to the selectivity of the supercritical fluid, the experimental design of the present invention verifies that the active matters of the functional ingredients in sorghum distillery residue, with high purity and low toxicity, can be obtained by using a supercritical carbon dioxide fluid to conduct the extraction, and can be appropriately used in the application to cosmetic and skincare products.

The present invention makes good use of the waste generated after the sorghum distillation and brewing process of the sorghum liquor, and hopes to transform the sorghum distillery residue waste into a usable matter. Not only is the environmental pollution thereof reduced, but also the economic value thereof is increased. Due to environmental awareness and increasingly serious tainted goods problems, the safety of skincare products and cosmetics which directly contact human skin cannot be ignored. In view of the fact that most of the common skin care products and cosmetics contain preservatives and artificial flavors and pigment, etc., if extraction and purification in the process of raw material synthesis are incomplete, long term use may cause an accumulation of chemicals in human body, resulting in skin damage such as allergy and irritation. Thus, customers are more advocating natural skincare products. Therefore, the present invention expects to develop cosmetic and skincare products that can be used with ease by customers.

The present invention researches and develops a more natural and environmental-friendly process for extracting the functional ingredients from the sorghum distillery residue. From experimental results, amounts of total polyphenols of extracts obtained by the solvent extraction method are about 207.46 mg/100 g SDR to 298.87 mg/100 g SDR, and an amount of total polyphenols of an extract obtained by the supercritical carbon dioxide fluid extraction is approximately 27.22 mg/100 g SDR. Previous studies have confirmed that the polyphenols have antioxidative capacity and inhibitory effects on tyrosinase, and there are no precedents for usage in cosmetics and skincare products. The optimal concentration for cosmetics and skincare products is found by using the extracts. The optimal amount of the total polyphenols, from the distillery residue, in cosmetics compositions is 0.001% to 0.02%, and the antioxidative capacity reaches more than 50% when the content thereof in cosmetic compositions is 0.02%.

### BRIEF DESCRIPTION OF THE DRAWINGS

The present invention will become more readily apparent to those ordinarily skilled in the art after reviewing the following detailed description and accompanying drawings, in which:

FIG. 1 shows the analytical results of the determination of the ability to scavenge DPPH (1,1-Diphenyl-2-picrylhydrazyl) free radicals;

FIG. 2 is an analytical result of MTT cell viability;

FIG. 3 is an analytical result of activity of intracellular tyrosinase; and

FIG. 4 is an analytical result of melanin production reduction.

### DETAILED DESCRIPTION OF PREFERRED EMBODIMENTS

The preparation of the sorghum distillery residue extracts and the application thereof in whitening skin care products will now be described with reference to the following embodiments. The embodiments are chosen and described in order to best explain the principles of the invention and its best mode practical application, thereby to enable persons skilled in the art to understand the invention for various embodiments and with various modifications as are suited to the particular use or implementation contemplated. It is intended that the scope of the invention be defined by the claims appended hereto and their equivalents in which all terms are meant in their broadest reasonable sense unless otherwise indicated. Any advantages and benefits described may not apply to all embodiments of the invention. It should be appreciated that variations may be made in the embodiments described by persons skilled in the art without departing from the scope of the invention as defined by the following claims.

### Embodiment 1—the Preparation of the Sorghum Distillery Residue Extracts

(1) Sorghum distillery residue: Samples are treated at 100° C.-120° C. by a continuous fluidized bed dryer and then are grinded by a pulverizer.

(2) Solvent extraction from sorghum distillery residue: 20 g of the sorghum distillery residue are weighed and put into conical flasks, and then a one-hour of stirring extraction is conducted with 200 ml pure water (75° C.) and 200 ml pure water (90° C.), respectively. At the end of the extraction, the volumes of filtrates are quantified after filtration with filter papers.

(3) Supercritical carbon dioxide fluid extraction from sorghum distillery residue: the supercritical fluid extraction of the present invention is conducted by using a 250 ml extraction tank. The three extractions take 93.51 g, 98.85 g, and 98.85 g of sorghum distillery residue powder, respectively. Carbon dioxide is turned on to enter the pipeline. A back pressure regulator is adjusted to control system pressures (three extraction pressures being 170 bar, 250 bar, and 300 bar, respectively), and the temperature in the extraction tank is adjusted (this time using a temperature of 40° C.). The solvents used are supercritical carbon dioxide and ethanol. Timing is started after the pressure and temperature reach the experimental set values. The samples are first soaked in the extraction tank for 2 hours for static extraction using ethanol and a carbon dioxide supercritical fluid. After that, dynamic extraction is conducted with carbon dioxide at a flow rate of 0.2 L/hr and ethanol at a flow rate of 0.4 L/hr for 2 hours. The samples collected in two separation tanks are mixed homogeneously and stored for use.

### Embodiment 2—Analysis of the Content of Total Polyphenolic Compounds (TPC)

(1) Preparation of Standards and Reagents:

Preparation of the standards: This experiment can use a conventional method with gallic acid (Acros Organics) as the standard. 0.1 g of anhydrous gallic acid are weighed and homogeneously dissolved in 100 ml pure water by ultrasonic oscillation for making the concentration of the standard be 1000 ppm. After that, according to the volumes shown in the following table, pure water is added to serially dilute the standard for different concentrations.

| | Concentration (mg/L); ppm | | | | | |
|---|---|---|---|---|---|---|
| | 0 | 10 | 20 | 30 | 40 | 50 |
| 1000 ppm standard sample (mL) | 0 | 1 | 2 | 3 | 4 | 5 |
| Pure water (mL) | 100 | 99 | 98 | 97 | 96 | 95 |

Preparation of the reagents: (a) Folin-ciocalteus phenol reagent is diluted 10-fold by pure water and reserved for use; and (b) 7.5% sodium carbonate (Na₂CO₃) aqueous solution.

(2) Experimental procedure: The standards and samples are prepared with pure water to appropriate concentrations, and then 1 ml of each is respectively taken and added to 5 ml of the 10-fold diluted Folin-ciocalteus phenol reagent. After mixing evenly, avoid light and stand for 5 minutes. Then, add 4 ml of 7.5% sodium carbonate aqueous solution, mix evenly, and then avoid light and stand for 1 hour, and centrifuge. And then determine their absorbance at 765 nm by a spectrophotometer. A linear regression analysis is performed with the concentrations of the standards as the abscissa and the absorbance as the ordinate. The linear regression analysis can be used only when the standard curve coefficient of determination (R²) is 0.999 to 1. Then, the absorbance value of the samples is brought into the standard curve to calculate the content of the total polyphenolic compounds. The extracted content of the total polyphenolic compounds is expressed in milligrams per 100 g of sorghum distillery residue (mg/100 g SDR).

(3) Experimental Results:

Table 1 shows the results of analysis of the content of the total polyphenols. Temperature does no obviously affect the result content when pure water is used for extraction. The amounts of the total polyphenols extracted by a supercritical fluid extraction manner are significantly lower than the amounts by water extraction. Since carbon dioxide with lower polarity is used as the solvent, and most of the polyphenolic compounds are with higher polarity because of containing hydroxyl group, their compatibility with less polar carbon dioxide is lower.

TABLE 1

| | the amount of the total |
|---|---|
| | polyphenols (mg/100 g SDR ) |
| Extraction with 90° C. pure water | 227.3 ± 3.7 |
| Extraction with 75° C. pure water | 207.5 ± 1.2 |
| Combined samples from three | 27.2 ± 0.39 |
| supercritical fluid extractions | |

### Embodiment 3—Determination of Ability to Scavenge DPPH (1,1-Diphenyl-2-picrylhydrazyl) Free Radicals

(1) Reagent preparation: 100 ml of ethanol is added to 0.004 g of DPPH, for a concentration of 40 ppm, which is homogeneously dissolved, protected from light and stored for use, and stored for no longer than half a day.

(2) Experimental procedure: prepare 200 ppm of each extract, which is then diluted sequentially to 100 ppm, 50 ppm, 25 ppm, and 12.5 ppm with the solvent used for extraction. Add 6 ml of DPPH to 1 ml of each sample. After preparation, the final concentrations of the samples are 28.6 ppm, 14.3 ppm, 7.1 ppm, 3.6 ppm, and 1.8 ppm. Mix homogeneously, then protect from light and stand for 40 minutes. Determine their absorbance at 517 nm by a spectrophotometer. The percentage of DPPH free radical scavenging effect of each extract is calculated according to the following formula.

$$Percentage of DPPH free radical scavenging effects ( \% ) scavenging rate = ( 1 - \frac{A 517 nm sample}{A 517 nm blank} ) \times 100 \%$$

(3) Experimental Results:

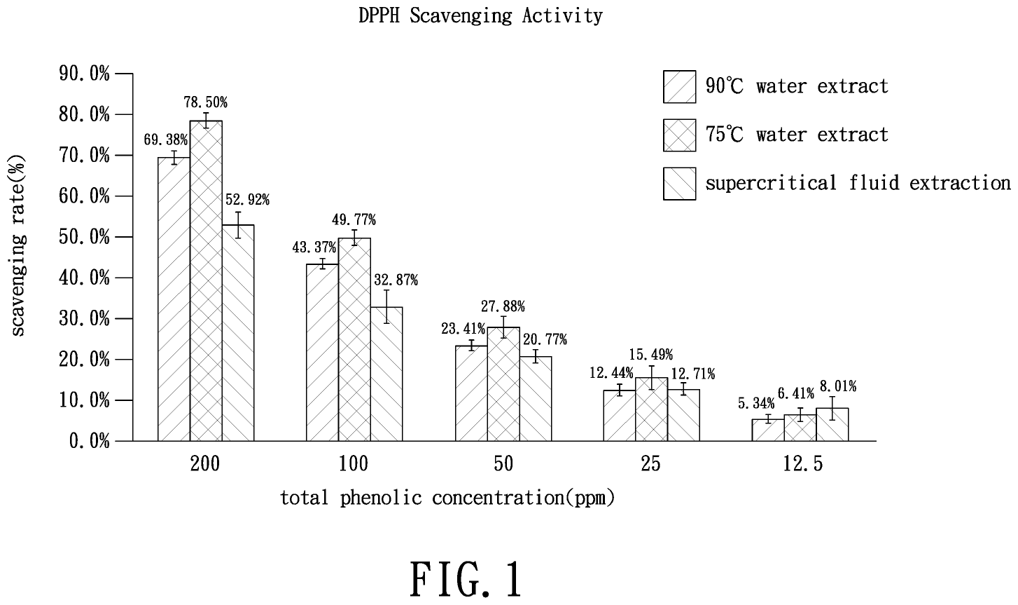

Since the substances contained in the extracts are more complicated and phenolic compounds are highly related to antioxidative capacity, experiments for the determination of DPPH free radical scavenging ability are based on the concentrations of the total polyphenols. FIG. 1 shows analytical results for experiments determining DPPH (1,1-Diphenyl-2-picrylhydrazyl) free radical scavenging ability, and the abscissa is the amount of the total polyphenolic compounds. FIG. 1 shows that the scavenging ability decreases as the extract concentration decreases, and is best with 75° C. water extract and 90° C. water extract, which have scavenging ability of approximately 78.5% and 69.3% at a concentration of 200 ppm (final concentration being 28.6 ppm) of total polyphenols from distillery residue.

The major substances with free radical scavenging ability in sorghum distillery residue are total polyphenolic compounds. The experimental results show that DPPH free radical scavenging ability of the extracts is positively related to the total polyphenolic compounds. From the experimental results, it can be seen that the extract samples from distillery residue can be used for anti-aging and anti-oxidation applications.

### Embodiment 4—Cell Viability Analysis

(1) Experimental Principle

According to a characteristic that tetrazolium in MTT reagents is reduced in living cells by dehydrogenase in mitochondria to generate bluish purple Formazan crystals, a relative proportion of surviving cells can be determined. The Formazan crystal has maximal absorbance at a wavelength of 570 nm. Therefore, the survival rate of cells is calculated by measuring the absorbance value.

(2) Experimental method:

Melanoma cells are implanted in a 24-well culture plate (5×10⁴ cells/well). After 24 hours of culture, cells are treated by the extract samples, from the sorghum distillery residue, with final concentrations of 10, 20, and 30 ppm for 24 hours. Each well is washed once by PBS (phosphate buffer saline), an MTT solution is added thereto, and then kept at 37° C. After 4 hours, Dimethyl Sulphoxide (DMSO) is used to solubilize the formazan product formed by the reduction of MTT by dehydrogenase in cell mitochondria. The absorbance (OD₅₇₀) of the products is determined by an enzyme-linked immunosorbent assay (ELISA) analyzer. The more cells survive, the deeper purple the color of the product.

Cell viability formula (%)=100−[(A−B)/A×100%]

A: OD₅₇₀ absorbance value of a control group

B: OD₅₇₀ absorbance value of a sample group

(3) Experimental Results:

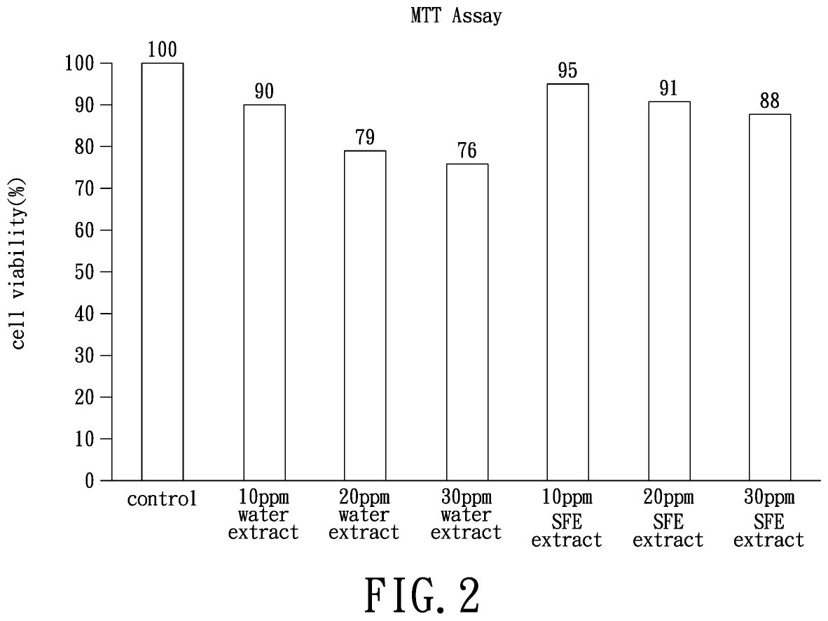

FIG. 2 shows results of cell viability analysis for water extraction and supercritical fluid extraction. The final concentrations of the total polyphenols from distillery residue are 10 ppm, 20 ppm, and 30 ppm, respectively. As shown in the experimental results, the supercritical fluid extracts from the same distillery residue show better cell viability. All extract samples show cell viability of more than 70%. From the experimental results, it can be concluded that the extract samples from the distillery residue have no toxic effect on the cells.

### Embodiment 5—Analysis of Activity of Intracellular Tyrosinase

(1) The Principle of the Analysis of Activity of Intracellular Tyrosinase

Using a freeze-thaw cycle method, cells are frozen at −80° C. for a period of time (30 minutes) such that the water inside the cells forms ice crystals. Then, the cells are thawed at room temperature for 30 minutes to recover the ice crystals into water. By this process, the cells are broken and the intracellular substances, including the tyrosinase to be measured, are released. The catalytic receptor (such as L-dopa) of the tyrosinase is added, and the relative activity of the tyrosinase can be deduced by analyzing the produced amount of its product.

(2) Analytical Method for Activity of Intracellular Tyrosinase

By measuring inhibition rates of autoxidation of dopachrome, it is determined whether the extracts from the distillery residue can inhibit tyrosinase activity and reduce melanin formation. B16F10 melanoma cells are implanted in a 24-well culture plate with a density of 5×10⁴ cells/ml and cultured in a culture medium containing 100 nM of α-MSH. After it is placed in a 37° C., 5% CO₂ incubator for 24 hours, distillery residue SFE is added to continue processing for another 24 hours. Then, 100 μl of 1% (V/V) Triton X-100/50 mM sodium phosphate buffer (pH 6.8) and 10 mM PMSF are added into each well and kept at −80° C. for 30 minutes. After it is brought to room temperature for 30 minutes and centrifuged at 12000 r.p.m. for 30 minutes, 80 μl of the supernatant is removed and placed in a 96-well plate, and a 1 mg/ml dopa solution is added into each well. OD₄₉₀ absorbance values are measured every 10 minutes for one hour in a row. Finally the tyrosinase inhibition rate is calculated.

inhibition rate formula (%)=(A−B)/A×100%

A: OD₄₉₀ absorbance of the control group

B: OD₄₉₀ absorbance of the sample group

(3) Experimental Results:

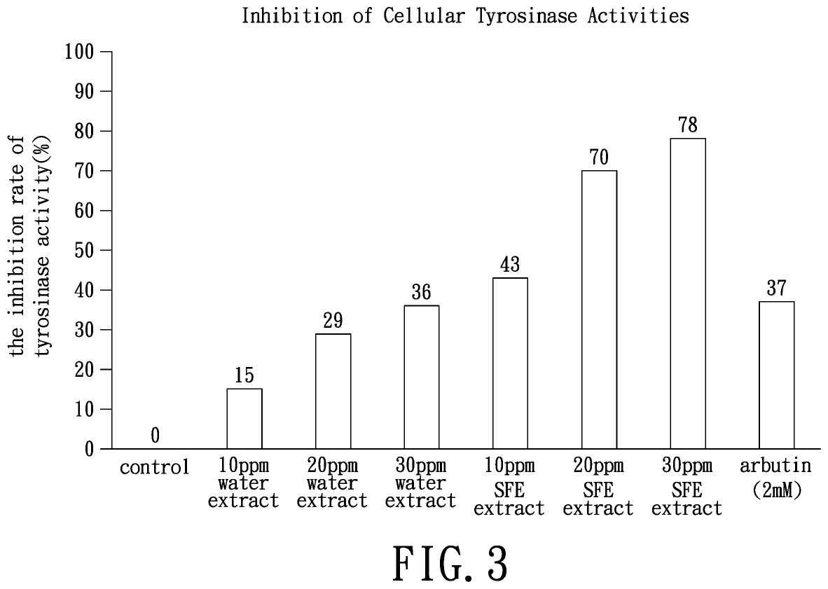

FIG. 3 shows analytical results of activity of intracellular tyrosinase. The extracts from distillery residue by supercritical extraction have better tyrosinase inhibition rate. The inhibition rate reaches over 70% when the concentrations of total polyphenols from the distillery residue are 20 ppm and 30 ppm in solutions. From the experimental results, it can be seen that the extract samples from distillery residue can be used for application in whitening effectiveness.

### Embodiment 6—Analysis of Melanin Content

(1) Principles of Analysis of Melanin Content

High temperature and strong alkali are utilized to dissolve out intracellular melanin. The effect of treatment under various conditions on melanin content in B16F10 melanoma cells is analyzed based on measurement at a specific absorbance wavelength of 405 nm.

(2) Method for Analysis of Melanin Content

Cells (5×10⁴ cells/ml) are implanted in a 24-well culture plate and cultured in a culture medium containing 100 nM of α-MSH, and in an incubator (37° C., 5% CO₂) for 24 hours. Afterwards, the original culture medium is replaced with a culture medium containing the distillery residue SFE or kojic acid of different concentration (in the control group, a culture medium without the distillery residue SFE or kojic acid is only replaced with the original culture medium). After incubation for another 24 hours, remove the culture medium and wash with PBS twice. Then, add 100 μl of 1N NaOH (prepared with 10% DMSO) for treatment at 60° C. for 1 hour. Finally, draw 80 μl into a 96-well plate to measure the absorbance at a wavelength of 405 nm and calculate the relative content of melanin.

(3) Experimental Results:

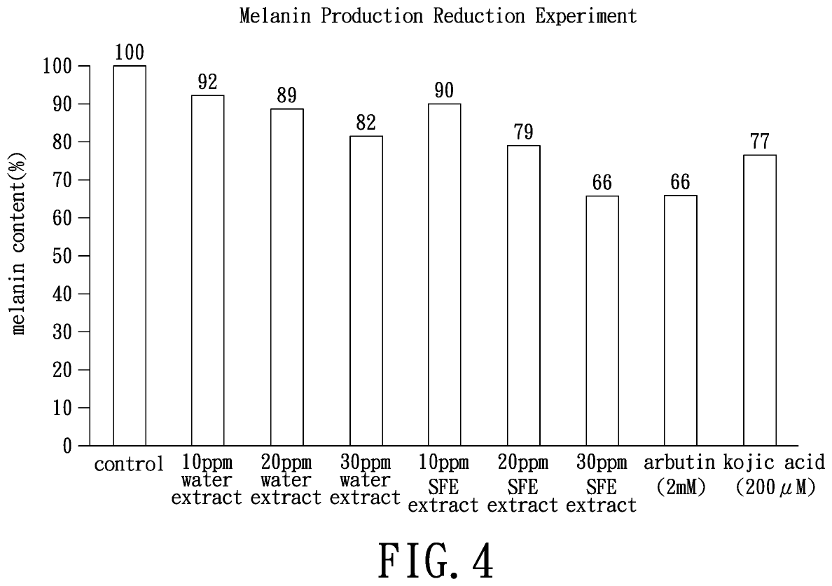

FIG. 4 shows the analytical results of the melanin content. Samples of extracts, from distillery residue, by water extraction, alcohol extraction and supercritical extraction all have effects of reducing the production of melanin. The final concentration of the total polyphenols, from distillery residue, at 30 ppm with supercritical extraction has better results. Based on the experimental results, it can be seen that the extract samples from distillery residue can be used for application in fading scars and spots.

### Embodiment 7—Compositions of Emulsions for Improving Skin Tones in Accordance with the Present Invention

| Ingredient | % w/w |
|---|---|
| Sorghum distillery residue extracts | 1.0 |
| Emulsifying agents ((glyceryl oleate (and) | 2-10 |
| polyglyceryl-3-polyricinoleate (and) olive oil | |
| (unsaturated fatty acid unsaponifiable)) | |
| Lipids (octyldodecanol, passiflora edulis seed oil, | 2-20 |
| oryza sativa bran oil, euterpe oleracea fruit oil, and | |
| camellia oleifera seed oil) | |
| Thickening agent (xantham gum) | 0.25 |
| Moisturizer (glycerol) | 1-10 |
| Perfume | appropriate amount |
| Preservatives (phenoxyethanol (and) caprylyl glycol) | 1.0 |
| Deionized water | up to 100 |

Brief description of production process: The steps are as follows.

(1) adding emulsifying agents ((glyceryl oleate (and) polyglyceryl-3-polyricinoleate (and) olive oil (unsaturated fatty acid unsaponifiable)) and lipids (octyldodecanol, Passiflora edulis seed oil, Oryza sativa bran oil, Euterpe oleracea fruit oil, and Camellia oleifera seed oil) into an oil phase pot, turning on stirring, increasing a temperature thereof to 80° C., and keeping the temperature constant for 5 minutes;

(2) stirring and homogeneously mixing deionized water, thickening agent (xantham gum) and moisturizer (glycerol), increasing a temperature thereof to 80° C., keeping the temperature constant for 5 minutes, and slowly pumped into an emulsification pot to be homogeneously emulsified for 30 minutes and decreasing the temperature thereof to 40° C.;

(3) adding sorghum distillery residue extracts, mixing homogeneously, and stirring evenly for 5 minutes;

(4) adding perfume, mixing homogeneously, and stirring evenly for 5 minutes; and

(5) adding preservatives (phenoxyethanol (and) caprylyl glycol), mixing homogeneously, stirring evenly for 20 minutes, and discharging when the temperature thereof decreases to 30° C.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A sorghum distillery residue extract, using Kinmen sorghum distillery residue as raw material and carbon dioxide as solvent, wherein sorghum distillery residue raw material is first statically extracted by ethanol and a carbon dioxide supercritical fluid for 2 hours and then continuously dynamically extracted by the carbon dioxide supercritical fluid for 2 hours, a moisture content of the used sorghum distillery residue is lower than 30% after drying, an operating pressure is between 150 bar and 350 bar, and a temperature is between 40° C. and 60° C.

2. A sorghum distillery residue extract, using Kinmen sorghum distillery residue as raw material, wherein extraction is conducted with pure water of a temperature of 50° C. to approximately 99° C. for 2 hours, and the extract has antioxidative activity, ability to inhibit tyrosinase and ability to reduce the content of melanin produced, and can be used as an additive for preparation of cosmetic or cosmeceutical compositions.

3. The extract according to claim 1, wherein optimal conditions for supercritical fluid extraction are: an operating pressure of 170 bar and a temperature of 40° C., an operating pressure of 170 bar and a temperature of 50° C., an operating pressure of 170 bar and a temperature of 65° C., an operating pressure of 250 bar and a temperature of 40° C., and an operating pressure of 300 bar and a temperature of 40° C.

4. The extract according to claim 1, wherein the extract has antioxidative activity, ability to inhibit tyrosinase, and ability to reduce the content of melanin produced, and can be used as an additive for preparation of cosmetic or cosmeceutical compositions.

5. The extract according to claim 2 or 4, wherein the extract is used as an additive for preparation of cosmetic or cosmeceutical compositions for skin aging prevention, whitening, tender whitening, fading spots, or bright skin.

6. The extract according to claim 5, wherein the extract is used as an additive for preparation of cosmetic or cosmeceutical compositions, and is present in an amount of 0.001 weight percent to 0.02 weight percent, based on a total weight of total polyphenols, from the distillery residue, of the compositions.

7. The extract according to claim 6, wherein the extract is used as an additive for preparation of cosmetic or cosmeceutical compositions, and the extract has better cytotoxicity, antioxidative capacity, and whitening ability when being in an amount of 0.001 weight percent to 0.003 weight percent, based on a total weight of total polyphenols, from the distillery residue, of the compositions.

8. The extract according to claim 7, wherein the extract is used as an additive for preparation of cosmetic or cosmeceutical compositions, and the extract, extracted with a supercritical fluid, has optimal cytotoxicity, antioxidative capacity and whitening ability when being in an amount of 0.003 weight percent, based on a total weight of total polyphenols, from the distillery residue, of the compositions.
